(19) United States
(12) Patent Application Publication
(10) Pub. No.: US 2005/0013869 A1
(43) Pub. Date: Jan. 20, 2005

(54) Sustained release formulation for carbamates and a method therefor

(76) Inventors: Cheng Shu Chaw, Singapore (SG); Yi-Yan Yang, Singapore (SG); Shabbir M. Moochhala, Singapore (SG); Donna Tan, Singapore (SG); Bin Zhao, Singapore (SG)

Correspondence Address: KLARQUIST SPARKMAN, LLP, 121 SW SALMON STREET, SUITE 1600, PORTLAND, OR, 97204, US

(21) Appl. No.: 10/622,316

(22) Filed: Jul. 18, 2003

(51) Int. Cl. A61K 9/22; A61K 9/50

(52) U.S. Cl. 424/501; 514/410

(57) ABSTRACT

The invention provides microparticles for sustained release formulation for physostigmine, pyridostigmine and other therapeutically active carbamates. The microparticles comprise the active compound and a biodegradable polymer such as polyester, poly(phosphate), poly(anhydride), poly(ortho ester), or mixture thereof. In one embodiment, the polymer is poly(d,l-lactide-co-glycolide). The desired release pattern of the active compound may be readily attained by varying the type and amount of the polymer used, including by using a mixture of two polymers, one of which is more hydrophobic. The invention also provides a method of preparing the microparticles and in one embodiment, the microparticles may be prepared by spray drying.

### FIELD OF THE INVENTION

The present invention relates to sustained release formulations of pharmaceutically active compounds, in particular, sustained release formulation of carbamates.

### BACKGROUND OF THE INVENTION

Organophosphosphorus (OP) pesticides and nerve gases bind to the esteratic site of acetylcholinesterase (AChE), an enzyme that hydrolyses a neurotransmitter known as acetylcholine (ACh) in the nervous system. OP pesticides bind to AChE in an irreversible manner, resulting in a subsequent accumulation of ACh in the nervous system at the synaptic cleft and myoneural junctions. Chronic exposure to OP pesticides affects both nicotinic and muscarinic ACh receptors. Classical symptoms of OP poisoning include cramps, nausea, vomiting, diarrhoea, etc. Acute poisoning occurs on exposure to high dose of nerve gases used in chemical warfare, which can lead to death.

One example of a class of therapeutic compounds is the carbamates, which are chemical compounds that bind reversibly with AChE. This class of compounds includes physostigmine, heptylphysostigmine, neostigmine, pyridostigmine, galanthamine, tetrahydroacridine, velnacrine and their pharmaceutically acceptable salts.

The carbamates temporarily occupy the catalytic site of AChE by forming a relatively unstable bond, thus preventing phosphorylation of the enzyme by OP agents (K. Tuovinen et al. (1999) Toxicology 134: 69-178; S. A. Miller et al. (1993) Pharmacol Biochem. Behav. 44(2): 343-347). Pyridostigmine, at a recommended dose of 30 mg orally every 8 hours, has been indicated for prophylaxis to protect soldiers against assault with nerve gases.

In addition, Myasthenia graves, a disorder characterized by defective neuromuscular transmission and muscular weakness due to formation of auto-antibodies to the acetylcholine receptor, has also been treated with carbamates such as pyridostigmine and neostigmine.

Physostigmine is moderately hydrophobic tertiary amine that can cross the human blood brain barrier. When compared to quaternary carbamates such as pyridostigmine, it has the advantage of protecting the CNS system on post-exposure to nerve agents. As well, an appropriate dose of physostigmine and other neuroactive AChE inhibitors such as tacrine and denepezil can be used to manage dementia (a common clinical feature of Alzhemier's disease), a syndrome characterized by deterioration of cognitive processes including memory, language and judgment etc.

However, physostigmine has a high first pass metabolism and a short elimination half-life of 12-40 minutes (K. Walter et al. (1995) Br. J. Clin. Pharmacol. 39: 59-63). To gain benefit following the oral administration, physostigmine has to be administered up to eight times daily (P. Hartvig et al. (1986) Acta Anesthesilo. Scand. 30: 177-182). Fine adjustment of drug input is required since this active compound is potent and may impair CNS performances. Dosing beyond the therapeutic window causes cholinergic symptoms similar to those of the poisoning (S. M. Somani and S. N. Dube (1989) Int. J. Clin. Pharmacol. Ther. Toxicol. 27(8): 367-387).

The strategy of using cocktail therapy as an antidote or prophylaxis treatment against poisoning of organophosphorus agents has been disclosed. Hille et al. in U.S. Pat. No. 6,114,347 describe using carbamic acid esters, such as physostigmine, in conjunction with other inhibitors of ACh receptors, such as scopolamine, L-hyoscyamine, benzatropine or benzetinmide. This reference describes using wax matrices and semi-permeable cellulose acetate membranes to effect controlled release of the active compounds.

Sommer et al., in U.S. Pat. No. 5,298,504, disclose the combination of physostigmine or pyridostigmine with either diazepam or clonazepam and an anti-cholinergic agent, Arpenal, Scyotrol, caramiphen or benactyzine. In U.S. Pat. No. 5,430,030, Sommer et al. describe a cocktail of pyridostigmine, diazepam and N-methyl-4-pipyridinyl phenylcyclopentanecarboxylate. Both of these cocktails are delivered in the form of a capsule containing three tablets: one as a normal release dosage, and one or two as a delayed release dosage.

Various physostigmine formulations, including transdermal delivery system (U.S. Pat. No. 5,939,095) and oral tablet formulations (U.S. Pat. Nos. 5,480,651 and 6,004,582), have been described. For oral delivery of physostigmine formulations, the use of tablet formulation for depot release is restricted as multiple doses are required for maintaining the efficacy of the treatment.

Madhat (U.S. Pat. No. 6,264,974) describes the incorporation of physostigmine into a composition to be administered buccally or sublingually for the treatment of Alzheimer's disease or nerve gas poisoning. This delivery system provides effective plasma concentrations of physostigmine that requires between 1 and 4 doses daily. However, this method involves the use of an aqueous carrier solution.

There are a few factors that need to be taken into consideration in formulating physostigmine, including the moderate water solubility of physostigmine, its fast dissolution kinetics and the ease at which it is hydrolyzed and oxidized in an aqueous medium (S. Rubnov et al. (2000) J. Pharm. Biomed. Ana. 18:939-945). In fact, active compounds that contain a carbamate functional group tend to be unstable in aqueous medium.

Sustained release formulations of pharmaceutically active compounds are designed to prolong the time that an active compound is maintained at an effective level in the blood or tissue. These formulations allow for less frequent doses to be administered, particularly where the active compound is unstable in the blood or tissue, or has a high clearance rate from the body. Other factors that may influence the choice of using a sustained release formulation for an active ingredient include the solubility, dissolution kinetics and rate of hydrolysis and oxidization of the active compound in an aqueous medium.

Thus, there is a need to design a sustained delivery system for physostigmine, pyridostigmine, and other carbamates.

### SUMMARY OF THE INVENTION

In one aspect, the invention provides microparticles comprising a pharmaceutically active carbamate and a biodegradable polymer. Microparticles of the invention effect sustained release of the carbamate and may therefore be used to prepare a sustained release formulation. In one aspect therefore, there is provided a sustained release formulation comprising microparticles according to the invention. In another aspect, there is provided a method of preparing microparticles of a pharmaceutically active carbamate comprising microencapsulating the carbamate with a biodegradable polymer.

The microparticles and sustained release formulation described herein are particularly advantageous for a relatively unstable pharmaceutically active compound, for example physostigmine, by protecting the drug from degradation. The invention also provides practical means of sustaining plasma level of compounds such as physostigmine and is therefore useful for reducing the dosing frequency of active compounds and providing a greater likelihood of compliance by the patient to a medication regiment.

In one particular embodiment, the microparticles comprising physostigmine as the active compound yielded very high encapsulation efficiency of physostigmine. A sustained release of physostigmine was observed for at least one week during the in vitro dissolution tests. In rats, the formulation comprising microparticles sustained plasma physostigmine level for up to 48 hours after a single oral administration. In comparison to non-microencapsulated physostigmine, the bioavailability of sustained release microencapsulated physostigmine was greatly improved without the induction of toxic side effects.

### BRIEF DESCRIPTION OF THE DRAWINGS

FIG. 1 shows typical SEM micrographs of microparticles prepared by spray drying. (a) PLA, (b) RG502, (c) PLGA 75:25. Polymer concentration: 3% w/v, initial physostigmine concentration: 10%w/w.

FIG. 2 shows the effect of inlet temperature on in vitro release profile of physostigmine-loaded PLA microparticles prepared by spray drying.

FIG. 3 shows in vitro release profiles of physostigmine-loaded microparticles prepared with various polymers by spray drying. Polymer concentration: 3 (w/v) %.

FIG. 4 shows variation of plasma physostigmine level after oral administration of suspension of physostigmine microparticles (4 mg/kg, n=6) or physostigmine solution (1 mg/kg, n=8).

### DETAILED DESCRIPTION

The invention provides microparticles comprising a pharmaceutically active carbamate and a biodegradable polymer. The microparticles effect sustained release of the carbamate and are therefore suitable for preparing a sustained release formulation. The invention therefore also provides a sustained release formulation comprising microparticles wherein the microparticles comprise a pharmaceutically active carbamate and a biodegradable polymer. The term “sustained release formulation” is used to describe a formulation that achieves a slower or a prolonged release of a drug over a period of time when compared to a conventional formulation. The term “pharmaceutically active carbamate” is used to describe a class of therapeutic AChE inhibitors or binding agents having a carbamate functional group. Included in this class are the compounds physostigmine, heptylphysostigmine, neostigmine, pyridostigmine, galanthamine, tetrahydroacridine, velnacrine, and their pharmaceutically acceptable salts.

Pharmaceutically acceptable salts will be apparent to one skilled in the art and include hydrochloride, hydrobromide, hydroiodide, bromide, sulfite, sulfate, bisulfate, nitrate, salicylate, citrate, tartarate, bitartarate, lactate, phosphate, malate, maleate, fumarate, succinate, acetate and pamoate salts.

In different embodiments, the biodegradable polymer is polyester, poly(phosphate), poly(anhydride) or poly (ortho ester), or a mixture thereof. The polyester in different embodiments may be poly(d,l-lactide-co-glycolide) (“PLGA”), poly(carprolactone) and polycarbonate. In one embodiment, the biodegradable polymer is a mixture of PLGA with other polyesters such as poly(caprolactone) and polycarbonate, or poly(anhydride), or poly(ortho ester) (“POE”).

The proportion of lactide to glycolide in PLGA may be between 0:100 and 100:0, between 50:50 and 85:15, between 50:50 and 75:25, and between 50:50 and 65:35. In specific embodiments, the lactide to glycolide content in PLGA may be 100:0, 50:50, 65:35, 75:25 and 85:15. Where the lactide to glycolide ratio is 100:00, the polymer is also known as poly(lactide) (“PLA”). Since microparticles comprising PLA may result in a very slow release of carbamate after initial burst release, use of PLA alone is generally not recommended unless a very slow release rate is desirable.

PLGA may have a wide range of average molecular weight (as determined, for example by gel permeation chromatography). In different embodiments, the average molecular weight is in the range of about 4,000 to about 100,000, and about 14,000 to about 42,000. In specific embodiments, the average molecular weight is about 14,600, about 15,000, about 41,800, about 45,400, about 83,200 and about 76,500. Since the molecular weight affects the apparent viscosity of the polymer solution, the suitable molecular weight range for PLGA and other polymers may vary depending on the solvent used in the preparation of microparticles. For example, when ethyl acetate is used, the average molecular weight less than about 60,000 for PLGA is preferred.

The effective concentration of carbamate present in the formulation of the invention may vary depending on the subject to whom the formulation will be administered and the intended treatment. Typically, microparticles will include the carbamate at a concentration range of about 1 (w/w) % to about 50 (w/w) %, preferably in the range of about 5 (w/w) % to about 20 (w/w) %. In one embodiment, the concentration is about 10 (w/w) %.

A biphasic pattern of release refers to an initial burst release of the active compound, followed by a sustained release of the active compound.

The degree of the initial burst release from microparticles can be controlled by varying the biodegradable polymer used. Also, inclusion of a more hydrophobic polymer, for example, in microparticles that contain PLGA, including a more hydrophobic polymer such as POE, have the effect of dampening the extent of the initial burst release. Therefore, by using a mixture of different polymers and by adjusting their ratio, the initial burst release can be controlled. In one embodiment, the microparticles comprise a first and second polymer wherein the second polymer is more hydrophobic than the first polymer. For example, poly(ortho esters) as a class are generally more hydrophobic than PLGA and in one embodiment, the biodegradable polymer is a mixture of PLGA and POE. As the same effect may be achieved provided a more hydrophobic polymer is included in the mixture, by way of an example, instead of or in addition to POE, PLGA may be used with any other biodegradable polymer that is more hydrophobic than PLGA, for example, with a more hydrophobic polyester including PLGA with higher lactide content and poly(caprolactone), or poly(anhydride).

The rate of release of active compound from the microparticles following the initial burst, in the case of a biphasic pattern of release, can also be controlled by varying the biodegradable polymer used. Generally, a polymer with greater hydrophobicity yields a slower sustained release due to slower water penetration. Thus, for example, a mixture of PLGA and a more hydrophobic polymer, may effect a slower sustained release of active compound than PLGA alone due to increased hydrophobicity. The hydrophobic polymer has been found to form an external coat on the microparticles when blended with PLGA, forming a hydrophobic layer that degrades more slowly in the aqueous environment of blood or tissue. Therefore, by using a mixture of polymers in which one polymer is more hydrophobic, for example a mixture of PLGA and a more hydrophobic polymer, and by varying the ratio of the two polymers, the desired sustained release rate may be achieved. A skilled person can readily determine the relative hydrophobicity of polymers, based on the physiochemical properties of the polymers or by measurement using standard techniques such as contact angle measurement.

Also, a higher glycolide content in PLGA increases the hydrophilic nature of the polymer. Therefore, microparticles with increased glycolide content in the PLGA may be used to achieve faster sustained release rates.

The desired release pattern therefore may be readily attained by varying the type and amount of polymer used.

The microparticles or formulation comprising microparticles may further comprise anti-cholinergic agents. In different embodiments, the anti-cholinergic agent may be, for example, scopolamine, arpenal, sycotrol-pipetabanate hydrochloride, caramiphen or benactyzine.

Conventional methods of microencapsulation can be used to prepare microparticles of the polymer and the carbamate including double emulsion, single emulsion and spray drying, using commercially available equipment. Accordingly, in another aspect, the invention relates to a method of preparing microparticles, and a sustained release formulation of a pharmaceutically active carbamate comprising microencapsulating the carbamate with a biodegradable polymer, which may include a polyester such as poly(d,l-lactide-co-glycolide), poly(phosphate), poly(anhydride), poly(ortho ester), or a mixture thereof.

Single emulsion (oil-in-water) techniques may not be effective for active ingredients containing a carbamate functional group. For example, the inventors have observed that single emulsion of physostigmine can result in loss of up to about 90% of the starting amount of physostigmine. This is likely due to the partitioning of the physostigmine into the aqueous phase during the emulsion process. Increasing the amount of dimethylsulfoxide (“DMSO”) used in the organic phase, for example dichloromethane (“DCM”), improves the level of capture of physostigmine.

Preferably, the microparticles are prepared by spray drying, since this method is simple, reproducible and easy to scale up, see F. Pavanetto et al. (1993) J. Microencapsulation 10(4): 487-497; M. D. L. Moretti et al. (2001) J. Microencapsulation 18(1): 111-121; P. O'Hara and A. J. Hickey (2000) Pharm. Res. 17(8): 955-961; B. Baras et al. (2000) Int. J. Pharm. 200(1): 133-145, the contents of all of which are hereby incorporated by reference. As well, the inventors have found this method can provide a very high encapsulation levels in which about 90% to 100% of a carbamate compound may be encapsulated in the microparticles.

To produce microparticles by spray drying, the carbamate and the polymer may be mixed in a solvent. A suitable solvent may depend on the carbamate that is to be formulated, but will preferably be a volatile organic solvent such as ethyl acetate, DCM, chloroform, tetrahydrofuran (“THF”), or a mixture thereof, which can dissolve the polymer and the active compound. The solvent may be any solvent or miscible co-solvent system which is volatile under fabrication conditions and which is able to dissolve both the active compound and the biodegradable polymer in a single phase. In one embodiment, the solvent is ethyl acetate.

The mixture of the solvent, active compound and biodegradable polymer is then spray dried. In one embodiment, the biodegradable polymer is first added to a solvent, preferably a volatile organic solvent, to a final concentration range of about 0.1 (w/v) % to 20 (w/v) %, preferably in the range of about 1 (w/v) % to 10 (w/v) %, more preferably in the range of about 2 (w/v) % to 6 (w/v) %, more preferably in the range of about 2 (w/v) % to 4 (w/v) %. In specific embodiments, the concentration is about 3 (w/v) % and 6 (w/v) %. Higher concentrations of polymer in the mixture can lead to the production of microparticles that have an irregular shape and that tend to aggregate. The desired amount of carbamate is then added prior to spray drying the mixture. As stated above, the carbamate may be added at a concentration range of about 1 (w/w) % to about 50 (w/w) %, preferably in the range of about 5 (w/w) % to 20 (w/w) %. In one embodiment, the carbamate is added at a concentration of about 10 (w/w) %. As well, an anti-cholinergic agent may be added to the mixture prior to spray drying.

Spray drying techniques are generally known in the art. Once sprayed dried, the microparticles obtained may be dried, for example in a desiccator, to remove any excess solvent.

The mixture may be spray dried at an inlet temperature of about 30° C. to 70° C., preferably about 50° C. to about 60° C. This inlet temperature corresponds to an outlet temperature of about 25° C. to about 60° C., and about 40° C. to about 50° C., respectively. Considerable burst release of drugs from the spray-dried microparticles is frequently observed due to high drug loading, small particle size and short diffusion path for surface associated drug molecules (H. Takada et al. (1995) PDA J Pharm Sci & Tech 49:180-184; P. Perugini et al. AAPS (2001) PharmSciTech; 2: Article 10). The inventors have found that the temperature at which the microparticles are formed by spray drying can affect the rate of release of the active compound. An increased inlet temperature results in a higher initial burst release. This may be due to the difference in porosity and/or size of microparticles produced at different temperatures. Accordingly, the release rate may be adjusted by varying the inlet temperature for spray drying the mixture.

The microparticles according to the invention can be formulated in a suitable manner for administration, including as a parenteral or oral formulation such that an effective amount of the active compound is combined in a mixture with a pharmaceutically acceptable vehicle.

For oral administration, the microparticles may be enclosed in hard or soft shell gelatin capsules, or it may be compressed into tablets, or it may be incorporated directly with food. For parenteral administration, solutions of the microparticles can be prepared in water suitably mixed with a surfactant such as hydroxypropylcellulose prior to administration. Dispersions can also be prepared in glycerol, liquid polyethylene glycols and mixtures thereof with or without alcohol, and in oils. Under ordinary conditions of storage and use, these preparations contain a preservative to prevent the growth of microorganisms. A person skilled in the art will know how to prepare suitable formulations. Conventional procedures and ingredients for the selection and preparation of suitable formulations are described, for example, in Remington's Pharmaceutical Sciences (2000 -20^{th} edition) and in The United States Pharmacopeia: The National Formulary (USP 26 NF21).

Formulations that contain biodegradable polymers that are sensitive to the gastric environment are preferably administered parenterally, for example by intramuscular or subcutaneous injection. The pharmaceutical forms suitable for injectable use include sterile aqueous solutions or dispersion and sterile powders for the extemporaneous preparation of sterile injectable solutions or dispersions.

The formulations comprising the microparticles may be used to treat or prevent organophosphorus or nerve gas poisoning, or injuries resulting therefrom and to treat a condition or disease responsive to treatment by a therapeutically active carbamate such as dementia, including Alzheimer's disease, and Myasthenia gravis. The terms “treat”, “treating” and the like means relieving, improving, or inhibiting a condition or disease or one or more symptoms thereof. The term “prevent”, “preventing” and the like means avoiding a condition or disease or one or more symptoms thereof.

Formulations can be prepared to contain an effective amount (meaning the amount sufficient to effect treatment or prevention of a condition or disease), for example, a suitable daily dose. The effective amount and suitable daily dose will vary depending on the active compound, the subject to be treated and the condition or disease and its severity and may be routinely determined by one skilled in the art. For example, for treatment of Alzheimer's disease, the formulation may include between about 25 mg and 50 mg of physostigmine in a single dosage. For treatment of organophosphate poisoning in humans, a typical formulation may contain between about 0.05 mg of physostigmine per kg of body weight for an intramuscular formulation, up to about 0.06 mg of physostigmine per kg of body weight for an oral formulation.

In one aspect, the invention therefore provides a method of treating or preventing organophosphorus or nerve gas poisoning or injuries resulting therefrom comprising administering a formulation according to the invention. In another aspect a method is provided for treating a condition or disease responsive to treatment by pharmaceutically active carbamate such as dementia, including Alzheimer's and Myasthenia gravis comprising administering a formulation according to the invention.

In another aspect, the invention provides use of microparticles and formulation according to the invention to treat or prevent organophosphorus or nerve gas poisoning or injury resulting therefrom and to treat a condition or disease responsive to treatment by pharmaceutically active carbamate such as dementia, including Alzheimer's disease and Myasthenia gravis. The invention also provides use of microparticles of the invention in the manufacture of a medicament to treat or prevent organophosphorus or nerve gas poisoning or injury resulting therefrom and to treat a condition or disease responsive to treatment by pharmaceutically active carbamate such as dementia, including Alzheimer's disease and Myasthenia gravis.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

The word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Physostigmine-Loaded PLGA Microparticles Prepared by Spray Drying

Materials: Physostigmine (eserine free base), PLGA 85:15 (Mw 76,500), PLGA 75:25 (Mw 83,200), PLGA 65:35 (Mw 45,400), PLGA 50:50 (Mw 41,800) were purchased from Sigma (St. Louis, Mo., USA). PLA (Mw 15,000) was purchased from Polysciences, Inc (Warrington, Pa. 18976, USA). RG 502 (Resomer®, PLGA 50:50, Mw 14,600) was obtained from Boehringer Ingelheim (Ingelheim, Germany). The molecular weights of all polymers were measured by a gel permeation chromatography (GPC) system consisting of a Waters 2690 separation module and a 410 RI detector (Waters, Milford, Mass., USA) with HR 4E and HR 5E columns (Waters, Milford, Mass., USA). Tetrahydrofuran (THF) (J. T. Baker, USA) was used as the mobile phase at a flow rate of 1.0 ml/min and polystyrenes (Polymer Laboratories Ltd, Amherst, Mass. 01002, USA) with various molecular weights were employed as calibration standards. Ethyl acetate was of analytical grade and obtained from Lab-Scan Analytical Sciences (Stillorgan, Co. Dublin, Ireland). All other chemicals and solvents were of analytical grade and used without further purification.

Fabrication of physostigmine-loaded microparticles: 3% to 6% (w/v) PLGA or PLA and 10% (w/w) physostigmine were dissolved in ethyl acetate. The solution was then spray-dried through the nozzle (0.7 mm in diameter) of a Buchi Mini 190 laboratory spray dryer (Buchi Laboratorium-Technik AG, Flawil, Switzerland). The inlet temperature was set at 50, 55 or 60° C. and thus the outlet temperature was about 40 to 50° C. The air flow rate was 700 NL/h. The rate of feeding and aspiration was fixed at 6 ml/min and 1 m³/min, respectively. Microparticles were collected and put in a desiccator under vacuum to remove residual solvents for at least 24 hours.

Determination of encapsulation efficiency of physostigmine: A fixed amount of spray-dried microparticles was dissolved in 25.0 ml DCM. Physostigmine entrapped in the microparticles was determined by UV-2501 PC UV-Vis Recording Spectrophotometer (Shimadzu, Japan) at 315 nm. The encapsulation efficiency was calculated as the ratio of actual and theoretical physostigmine content. Each sample was assayed in triplicate.

Morphology of the microparticles: The morphology of microparticles was examined using scanning electron microscopy (SEM) (JSM-5310LV Scanning Microscopy, JEOL).

In vitro release tests: 10 mg of microparticles were suspended in 1 mL PBS (pH 7.4). They were incubated at 37° C. At pre-determined time intervals, the suspension was centrifuged at 10,000 rev/min for 3 min, and the supernatant was taken out for the determination of physostigmine concentration. The incubation medium was replaced with fresh buffer.

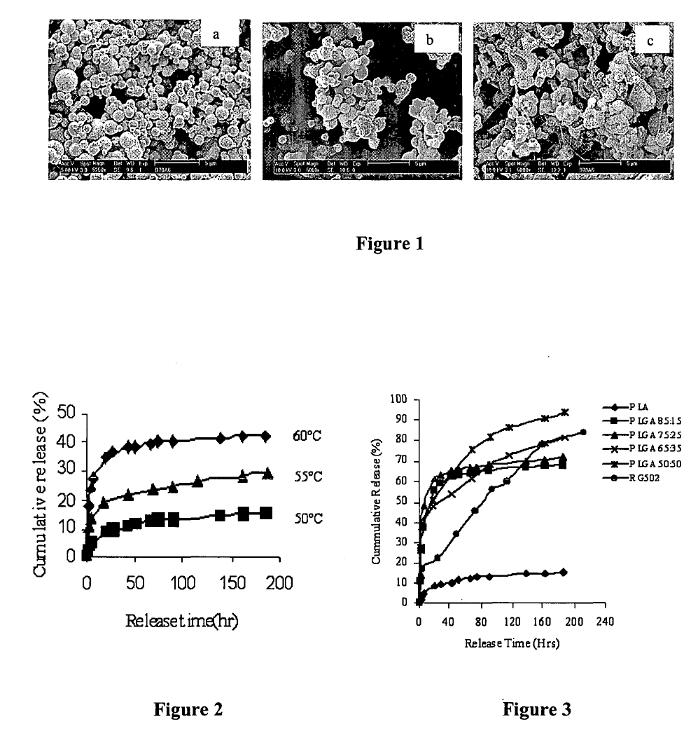

Results: More than 90% encapsulation efficiency of physostigmine was obtained using the spray drying technique (see Table 1). SEM micrographs revealed that spherical microparticles containing physostigmine with a smooth surface and narrow size distribution were yielded with PLA, PLGA 50:50, RG 502 and PLGA 65:35 (3% (w/v)). PLGA 85:15, PLGA 75:25 and PLGA 50:50 at a high concentration (6% (w/v)) produced microparticles with irregular shapes. Typical SEM scans of the microparticles are shown in FIG. 1.

TABLE 1
Encapsulation efficiency of physostigmine-encapsulated PLGA and
PLA microparticles fabricated by the spray drying process

| Polymer and its concentration | Inlet tempera- | Encapsulation efficiency |
|---|---|---|
| (% w/v) | ture (° C.) | (%) |
| PLA, 3 | 60 | 101 |
| PLA, 3 | 55 | 93 |
| PLA, 3 | 50 | 102 |
| PLGA 85:15, 3 | 50 | 102 |
| PLGA 75:25, 3 | 50 | 102 |
| PLGA 65:35, 3 | 50 | 96 |
| PLGA 50:50, 3 | 50 | 97 |
| PLGA 50:50, 6 | 50 | 93 |
| RG 502, 3 | 50 | 97 |

Fabrication temperature had an obvious effect on physostigmine release from PLA microparticles. An increased inlet temperature resulted in faster initial burst release of physostigmine (FIG. 2). This may be due to the difference in porosity and size of microparticles produced at different temperatures. Physostigmine release from the PLGA and PLA microparticles showed a biphasic pattern (FIGS. 2 and 3). In general, more hydrophilic polymers (i.e. having a higher content of glycolide) yielded a higher physostigmine release rate due to faster water penetration and polymer degradation. An increased inlet temperature resulted in faster physostigmine release. RG 502 microparticles demonstrated a low initial burst release and provided sustained physostigmine release over one week.

Physostigmine is unstable in an aqueous solution. It can easily hydrolyze and become eseroline, which is further oxidized to eserine brown. The stability of physostigmine in the in vitro medium at 37° C. was performed. The physostigmine degraded as a function of incubation time and about 40% physostigmine was degraded in 6 days (7% per day) (C. S. Chaw et al. (2003) Biomaterials 24(7): 1271-1277). From the in vitro release profile of physostigmine from RG502 microparticles, more than 80% of physostigmine was released over 7 days. Therefore, by using a microparticle system, the stability of the physostigmine is improved.

### Example 2

### In vivo Study of Physostigmine-Loaded RG502 Microparticles

Naked physostigmine solution (1 mg/kg) or physostigmine-loaded RG 502 microparticles suspension (10% drug loading) were fed intragastrically into the overnight-fasted rats (weight-280g) via a rigid dosing gavage needle into the posterior of the rat pharynx, directly into the stomach. Immediately before administration of the tablets and solutions, a 300 μL sample of blood was taken at what was t=0 hrs (pre-dose), through the exposed catheter. Subsequent samples were drawn from the catheter at intervals over a period of 48 hours after drug administration. After each withdrawal, an equal volume of 0.9% normal saline was injected back into the blood stream to minimize loss of body fluid. Water and food were available ad libitum in the metabolic cages. Each 300 μl volume of blood was collected in heparinised microcentrifuge tubes and centrifuged under 3000 g for 5 minutes at 4° C. to obtain the plasma. All plasma samples were stored at −70° C. in fresh heparinised microcentrifuge tubes. Physostigmine is extracted immediately from it prior to analysis by high performance liquid chromatography (HPLC) described by Zhao et al (2003) Journal of Chromatography B 784- 323-329). The WinNonlin® Version 3.2 (Pharsight Corporation, USA) was used to calculate the pharmacokinetic parameters based on the non-compartment model.

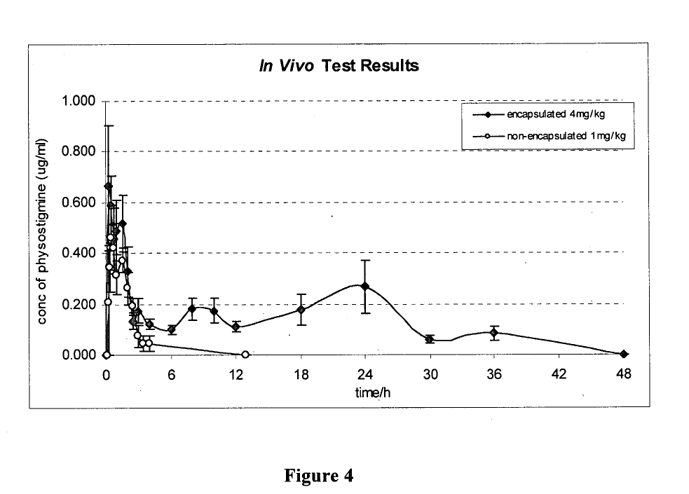

Results: The physostigmine-loaded RG502 microparticles were utilized as an oral dosage form for in vivo rat tests. The results showed that the formulation was capable of sustaining plasma physostigmine level up to about 48 hours (FIG. 4) and it increased the half-life of physostigmine by 15-fold without affecting the peak concentration and latency to peak concentration, as compared to the aqueous physostigmine solution (Table 2).

TABLE 2
Pharmacokinetics data for physostigmine oral solution (1 mg/kg dose) and
microparticle suspensions (4 mg/kg)
suspensions (4 mg/kg)

| | Sustained release formulation, | | | | | |
|---|---|---|---|---|---|---|
| | 4 mg/kg | | | Naked physostigmine, 1 mg/kg | | |
| Parameters | Mean | SD | SEM | Mean | SD | SEM |
| Tmax (hr) | 0.63 | 0.47 | 0.19 | 0.68 | 0.43 | 0.15 |
| Cmax (μg/mL) | 0.82 | 0.47 | 0.19 | 0.57 | 0.19 | 0.066 |
| Half life (hr) | 18.28 | 9.01 | 3.68 | 1.16 | 0.42 | 0.149 |
| AUC (hr*μg/mL) | 5.95 | 2.90 | 1.19 | 0.77 | 0.28 | 0.099 |
| Vd (mL/kg) | 13681.29 | 4272.22 | 1744.13 | 1584.37 | 480.60 | 169.92 |
| Cl (mL/hr/kg) | 633 | 364 | 14.5 | 998.86 | 312.68 | 110.55 |

1. Microparticles comprising a pharmaceutically active carbamate and a biodegradable polymer.

2. The microparticles of claim 1 wherein the biodegradable polymer is polyester, poly(phosphate), poly(anhydride), poly(ortho ester) or a mixture thereof.

3. The microparticles of claim 2 wherein the polyester is poly(d,l-lactide-co-glycolide), poly(caprolactone), polycarbonate or a mixture thereof.

4. The microparticles of claim 3 comprising a mixture of a first and a second polymer wherein the second polymer is more hydrophobic than the first polymer.

5. The microparticles of claim 4 wherein the first polymer is poly(d,l-lactide-co-glycolide) and the second polymer is a polyester, poly(anhydride) or poly(ortho ester).

6. The microparticles of claim 3, wherein the carbamate is physostigmine, heptylphysostigmine, neostigmine, pyridostigmine, galanthamine, tetrahydroacridine, velnacrine, or a mixture thereof.

7. The microparticles of claim 6 wherein the biodegradable polymer is poly(d,l-lactide-co-glycolide).

8. The microparticles of claim 7, wherein the carbamate is physostigmine.

9. The microparticles of claim 7, wherein the carbamate is pyridostigmine.

10. The microparticles of claim 8, wherein the poly(d,l-lactide-co-glycolide) has an average molecular weight range of about 4,000 to about 100,000.

11. The microparticles of claim 10, wherein the poly(d,l-lactide-co-glycolide) contains lactide and glycolide in a ratio of lactide:glycolide of 85:15, 75:25, 65:35 or 50:50.

12. The microparticles of claim 10, wherein the poly(d,l-lactide-co-glycolide) has an average molecular weight range of about 14 000 to 42 000.

13. The microparticles of claim 11, wherein the concentration of the polymer is about 2% to 6% w/v.

14. The microparticles of claim 13, wherein the concentration of the carbamate is about 10% w/w.

15. A sustained release formulation comprising microparticles wherein the microparticles comprise a pharmaceutically active carbamate and a biodegradable polymer.

16. The formulation of claim 15 which is an oral or parenteral preparation.

17. The formulation of claim 15 that provides sustained release of the carbamate for up to about 48 hours, wherein the carbamate is physostigmine and the polymer is poly(d,l-lactide-co-glycolide) containing lactide and glycolide in a ratio of 50:50 and the concentration of the carbamate is 10% w/w of the microparticles.

18. The microparticles of claim 8 that provide sustained release of the carbamate for at least one week, wherein the concentration of the carbamate is 10% w/w.

19. The formulation of claim 15 further-comprising an anti-cholinergic agent.

20. A method of preparing a sustained release formulation of a pharmaceutically active carbamate comprising microencapsulating the carbamate with a biodegradable polymer.

21. The method of claim 20, wherein the carbamate is physostigmine, heptylphysostigmine, neostigmine, pyridostigmine, galanthamine, tetrahydroacridine, velnacrine, or a mixture thereof.

22. The method of claim 21 wherein the biodegradable polymer is polyester, poly(d,l-lactide-co-glycolide), poly(phosphate), poly(anhydride), poly(ortho ester), of a mixture thereof.

23. The method of claim 22 wherein the polymer is poly(d,l-lactide-co-glycolide).

24. The method of claim 23 wherein the carbamate is physostigmine.

25. The method of claim 24, wherein the step of microencapsulation is effected by spray drying.

26. The method of claim 25, comprising the step of mixing the carbamate and the polymer in a volatile organic 'solvent prior to spray drying.

27. The method of claim 26 wherein the solvent is ethyl acetate.

28. The method of claim 27, wherein the spray drying is performed at an inlet temperature of about 50° C. to 60° C.
